(19) United States
(12) Patent Application Publication
(10) Pub. No.: US 2025/0288925 A1
(43) Pub. Date: Sep. 18, 2025

(54) METHOD OF SEPARATING SOLIDS FROM A SOLUTION DERIVED FROM PLASMA

(71) Applicant: CSL BEHRING (AUSTRALIA) PTY LTD, Melbourne (AU)

(72) Inventors: Karl MCCANN, Melbourne (AU); Zhenghui XU, Melbourne (AU); Per HANSEN, Melbourne (AU); Jiaxi ZHANG, Melbourne (AU)

(73) Assignee: CSL BEHRING (AUSTRALIA) PTY LTD, Melbourne (AU)

(21) Appl. No.: 18/869,203

(22) PCT Filed: May 31, 2023
(86) PCT No.: PCT/AU2023/050459

§ 371 Date: Nov. 25, 2024

(30) Foreign Application Priority Data
May 31, 2022 (AU) 2022901481

(51) Int. Cl. B01D 21/26; A61K 35/16; B04B 1/20; B04B 13/00

(52) U.S. Cl. CPC B01D 21/262; A61K 35/16; B04B 1/20; B04B 13/00; B01D 2221/10

(57) ABSTRACT

A method (100) of separating solids from a solution (46) derived from blood plasma is disclosed. The method (100) comprises feeding (102) the solution (46) into a decanter centrifuge (10) at a feed rate. The method (100) further comprises centrifuging (104) the solution (46) using the decanter centrifuge (10) to produce a centrate (48) having a reduced solids content with respect to the solution (46) fed into the decanter centrifuge (10) such that a mass of the centrate (48) is at least 75% of a mass of the liquid phase of the solution (46) fed into the decanter centrifuge (10).

### TECHNICAL FIELD

The present disclosure relates, generally, to a method of separating solids from liquids, and, more particularly, to a method of separating solids from a solution derived from blood plasma.

### BACKGROUND

Advances in the understanding of the function of blood plasma proteins and the deficiencies involved in a variety of blood disorders, combined with improvements in techniques for storage of the major protein components of human blood, have resulted in increased utilisation of specific sub-fractions of human blood, in particular the cellular components (erythrocytes, thrombocytes and leukocytes) and plasma protein fractions (albumins, fibrinogen and globulins including euglobulins, pseudoglobulins, alpha-globulins, beta-globulins and gamma globulins, such as immunoglobulin G (IgG)), rather than whole blood, for therapeutic purposes.

The plasma protein fraction of human blood, in particular, is of enormous value to the pharmaceutical industry in the production of therapeutics for the treatment of fibrinogenic, fibrinolytic and coagulation disorders and immunodeficiencies, for example haemophilia, von Willebrand's disease and fibrinogen deficiency, amongst others.

Blood plasma fractions are formed from blood plasma fractionation processes such as the Cohn process, the Kistler and Nitschmann process or variations of these processes. These industrial scaled cold ethanol fractionation methods enable multiple plasma proteins to be extracted from the one plasma source. Such processes generally involve frozen plasma (batch sizes in the range of 1000-15000 kg) being thawed to form an albumin rich cryosupernatant and a cryoprecipitate. The cryoprecipitate contains valuable coagulation factors that are subsequently separated from the cryosupernatant. In the Cohn or Kistler and Nitschmann processes, the cryosupernatant may be optionally exposed to an initial low ethanol (typically 8%) precipitation stage to remove Fibrinogen. Again, the precipitate (Fraction I) is removed and can be used to make other products such as Fibrinogen. Adsorption steps using ion-exchange or affinity resins are also optionally conducted across either of these two intermediate fractions to extract other proteins (e.g. Prothrombin complex; Antithrombin III; C1 esterase inhibitor). Subsequently, the albumin is extracted from the Supernatant I by raising the ethanol concentration to about 25% at about pH 6.9 for the Cohn method or about 19% at about pH 5.85 for the Kistler and Nitschmann method, the immunoglobulins are precipitated (Fraction (I+)II+III or Precipitate A) while the albumin remains in solution (Supernatant (I+)II+III or Filtrate A). Albumin is then isolated from the majority of the other plasma contaminants (mainly a and β globulins), which are precipitated by the further addition of ethanol to a final ethanol concentration of about 40% (Fraction IV). In a final step, the albumin is itself precipitated near its isoelectric point. The precipitate paste (Fraction V) can be held frozen before further processing. It is important to recognise that these processes have some adaptability and have been optimised over the years to suit each manufacturer's product portfolio. An example of this would be the presence or absence of an additional Cohn fractionation step (Fraction IV-1) following Fraction (I+)II+II step that can be used to extract alpha-1-antitrypsin. Another example is the use of Fraction IV-4 derived from Fraction II+III or Fraction (I+)II+III to extract hemopexin.

Separation processes are required to purify or separate larger solid contaminants from blood plasma fractions in order to further process blood plasma fractions to obtain the required protein component. A filter press is commonly used to separate the solid and liquid phases. The filtration process takes place in the filter plates of the filter press, where several filter plates are joined to one another to form a filter plate assembly. With the aid of a closing cylinder, usually a hydraulic closing cylinder, pressure is applied to the filter plate assembly in order to guarantee the necessary leak-tightness between the individual filter plates. Each of the filter plates has a filter area covered with a filter media, where the resuspended plasma fraction to be filtered is pressed into the filter chamber formed between two filter plates and against the filter media. Application of the product to the filter press allows for the liquid to pass through the filter media, whilst the solids are retained on the filter media. The filtrate between the filter media and the filter area is then carried off, and the filter cake remains in the filter chamber. The solids, which form a filter cake is harvested from the filter press when the filter plates are separated from one another.

However, the operation of the filter press is labour intensive and time consuming. Further, filter presses have a large footprint and therefore require large spaces for storage and operation.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

In an aspect of the present disclosure, there is provided a method of separating solids from a solution derived from blood plasma, the method comprising: feeding the solution into a decanter centrifuge at a feed rate; and centrifuging the solution using the decanter centrifuge to produce a centrate having a reduced solids content with respect to the solution fed into the decanter centrifuge, wherein the decanter centrifuge comprises: a scroll rotating at a scroll speed, a bowl rotating at a bowl speed to produce a bowl g-force, wherein the bowl speed is different to the scroll speed, thereby defining a speed differential that determines the rate at which the solid phase is conveyed by the scroll, and a weir on an internal surface of the bowl, the weir having a dimension that defines a pond depth, and wherein one or more of the feed rate, the bowl g-force, the speed differential, and the weir dimension are selected such that a mass of the centrate is at least 75% of a mass of the liquid phase of the solution fed into the decanter centrifuge.

The solution may comprise a blood plasma fraction. The solution may comprise a buffer. The buffer may comprise sodium acetate, water for injection, or a phosphate. The solution may have an extraction ratio of kilograms of blood plasma fraction to litres of buffer of from about 1:2 to about 1:10.

The solution may comprise hemopexin. The solution may comprise albumin. The solution may comprise immunoglobulin G. The method may further comprise adding octanoic acid to the solution or the centrate for delipidation.

The solution may comprise a filter aid. The feed rate of the solution may be up to about 10,000 kilograms per hour depending on the bowl size. The bowl g-force may be from about 1000 g to about 4000 g. The speed differential may be from about 1 revolution per minute to about 200 revolutions per minute. The weir dimension may be selected such that the pond depth is from about 1 millimetres to about 100 millimetres.

The mass of the centrate may be at least 75% of the mass of the solution fed into the decanter centrifuge. The mass of the centrate may be at least 90% or even at least 95% of the mass of the solution fed into the decanter centrifuge. A turbidity of the centrate will depend on the solution being processed. In some examples, the turbidity of the solution may be less than 400 nephelometric turbidity units (NTU), or in some embodiments less than 35NTU. However, it will be appreciated that where the solution is immunoglobulin G, the turbidity values may be higher when octanoic acid is added.

The method may further comprise washing the solids during the centrifuging. The method may further comprise filtering the centrate.

The filtering the centrate may comprise using a depth filter. The depth filter may have a retention rating of 15 microns or finer. The centrate may be produced such that the centrate is filterable with a throughput of at least 15 kg blood plasma fraction per square metre of the depth filter. A turbidity of the filtered centrate using the depth filter may be less than 150 nephelometric turbidity units. The mass of the filtered centrate using the depth filter may be at least 95% of the mass of the centrate. The method may further comprise adding a filter aid to the centrate prior to the using the depth filter to filter the centrate.

The filtering the centrate may comprise using a membrane filter. The membrane filter may be a 1 micron filter or finer. The filtering the centrate may comprise using a combination of a depth filter and a membrane filter. The filtrate may be produced with a throughput of at least 300 litres per square metre of the membrane filter. A turbidity of the filtrate using the membrane filter may be less than 50 NTU. The mass of the filtrate using the membrane filter may be at least 99% of the mass of the solution applied to the membrane filter.

In another aspect of the present disclosure, there is provided a method of separating solids from a solution derived from blood plasma, the method comprising: feeding the solution into a decanter centrifuge at a feed rate; and centrifuging the solution using the decanter centrifuge to produce a centrate having a reduced solids content with respect to the solution fed into the decanter centrifuge, wherein the decanter centrifuge comprises: a scroll rotating at a scroll speed, a bowl rotating at a bowl speed to produce a bowl g-force, wherein the bowl speed is different to the scroll speed, thereby defining a speed differential, and a weir on an internal surface of the bowl, the weir having a dimension that defines a pond depth.

In another aspect of the present disclosure, there is provided a blood plasma product produced using any embodiment of the method described above.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the disclosure will now be described by way of example only with reference to the accompanying drawings in which:

FIG. 1 shows a sectional schematic of an embodiment of a decanter centrifuge;

FIG. 2 shows a cutaway sectional schematic of the decanter centrifuge shown in FIG. 1 with different weir embodiments of the decanter centrifuge;

FIG. 3 shows a flowchart of an embodiment of a method of separating solids from a solution derived from blood plasma using the decanter centrifuge shown in FIG. 1 to produce a centrate;

FIG. 4 shows a flowchart of another embodiment of the method shown in FIG. 3;

FIG. 5 shows a line graph of experimental results of centrate turbidity versus flow rate under different bowl g-forces from applying the method shown in FIG. 3 to the solution;

FIG. 6 shows a line graph of experimental results of centrate turbidity versus residence time under different bowl g-forces from applying the method shown in FIG. 3 to the solution;

FIG. 7 shows a line graph of experimental results of centrate liquid recovery versus flow rate under different bowl g-forces and speed differentials from applying the method shown in FIG. 3 to the solution;

FIG. 8 shows a line graph of experimental results of centrate turbidity versus bowl g-force under different feed rates and speed differentials from applying the method shown in FIG. 3 to the solution;

FIG. 9 shows a line graph of a particle size distribution analysis of the centrate produced by applying the method shown in FIG. 3 to the solution;

FIG. 10 shows a line graph of experimental results of throughput versus time under different depth filter media and optional filter aids from applying the method shown in FIG. 4 to the solution;

FIG. 11 shows a column graph of experimental results of throughput and turbidity under different depth filter media and optional filter aids from applying the method shown in FIG. 4 to the solution;

FIG. 12 shows a line graph of experimental results of throughput and flux versus time in comparing a depth filter and filter aid combination from FIGS. 10 and 11 to a mimicked filter press from applying the method shown in FIG. 4 to the solution;

FIG. 13 shows a line graph of experimental results of throughput and flux versus time under the depth filter media and filter aid combination from FIG. 12 and a first membrane filter from applying the method shown in FIG. 4 to the solution;

FIG. 14 shows a line graph of experimental results of throughput and flux versus time under the depth filter media and filter aid combination from FIG. 12 and a second membrane filter from applying the method shown in FIG. 4 to the solution;

FIG. 15 shows a line graph of experimental results of throughput and flux versus time under the depth filter media and filter aid combination from FIG. 12 by applying the method shown in FIG. 4 to another embodiment of the solution; and

FIG. 16 shows a line graph of experimental results of throughput and flux versus time under the depth filter media and filter aid combination from FIG. 12 by applying the method shown in FIG. 4 to another embodiment of the solution.

### DETAILED DESCRIPTION OF EXEMPLARY EMBODIMENTS

### General Terms

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

The term “and/or”, e.g., “X and/or Y” shall be understood to mean either “X and Y” or “X or Y” and shall be taken to provide explicit support for both meanings or for either meaning.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The term “about” as used herein means within 5%, and more preferably within 1%, of a given value or range. For example, “about 3.7%” means from 3.5 to 3.9%, preferably from 3.66 to 3.74%. When the term “about” is associated with a range of values, e.g., “about X % to Y %”, the term “about” is intended to modify both the lower (X) and upper (Y) values of the recited range. For example, “about 20% to 40%” is equivalent to “about 20% to about 40%”.

### Specific Terms

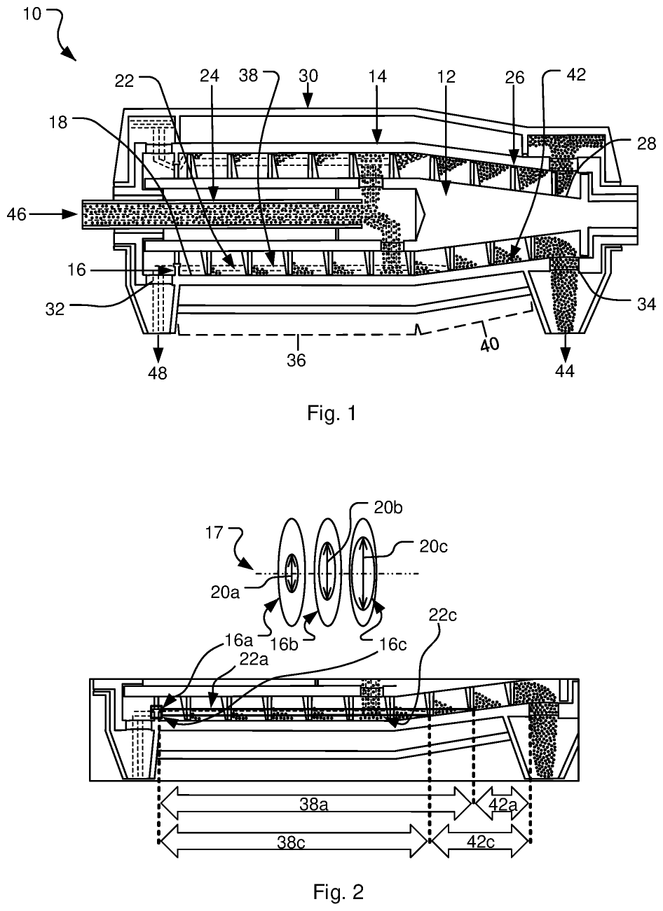

The term “recovery” or “recovery percentage” is used to refer to the mass of the centrate 48 as a percentage of the mass of the solution 46 undergoing centrifuging (FIG. 1). Recovery may be used as a performance indicator with the operating parameters of the decanter centrifuge varied to achieve a minimum recovery. It will be appreciated that the desired recovery will vary based on a variety of factors, for example the composition of the feed solution, the intended use of the final product and whether the centrate will undergo one or more further separation processes such as filtering.

The term “throughput” is used with regard to filtering processes and is defined as the total volume of the centrate filtered per unit surface area of the filter, represented as litres of the centrate per square metre of the filter (L/m²), and/or by total mass of blood plasma fraction per unit surface area of the filter (kg/m²). It will be understood that any suitable units may be used such as gallons of the centrate per square foot of the filter or pounds of the blood plasma fraction per square foot of the filter. Throughput may be used as a performance indicator of the filtration process(es), with higher throughput typically indicative of improved recovery, filter performance (e.g. the filter not being clogged or obstructed by solids). It will be appreciated the filter properties and operating conditions selected to provide a desired throughput.

The term “flux” is used with regard to filtering processes and is defined by the total volume of the centrate filtered per unit of time per unit surface area of the filter, represented by litres of the centrate per minute per square metre of the filter (L/min/m²). Flux may also be defined as throughput per unit of time. It will be understood that any suitable units may be used such as gallons of the centrate per second per square foot of the filter. Flux may be used as a performance indicator of the filtration process(es), with higher flux typically indicative of improved recovery and filter performance (e.g. the filter not being clogged or obstructed by solids). It will be appreciated the filter properties and operating conditions selected to provide a desired flux.

Method of Separating Solids from a Solution Derived from Blood Plasma

In the drawings, reference numeral 10 generally designates an embodiment of a decanter centrifuge 10 comprising a scroll 12 rotating at a scroll speed and a bowl 14 rotating at a bowl speed to produce a bowl g-force (FIG. 1). The bowl speed is different to the scroll speed, thereby defining a speed differential, that is, a difference in rotational speed between the scroll 12 and the bowl 14 which may be measured in revolutions per minute (rpm). The decanter centrifuge 10 also comprises a weir 16 on an internal surface 18 of the bowl 14, the weir 16 having a dimension that defines a pond depth of a pond 22 which may be measured in millimetres (mm). As shown in FIG. 2, weirs 16a, 16b, 16c can be selected having different dimensions 20a, 20b, 20c in order to provide the desired pond depth 22a, 22c.

The decanter centrifuge 10 comprises a feed tube 24, flights 26 provided on an external surface 28 of the scroll 12, a casing 30, a liquid outlet 32 and a solids outlet 34. The bowl 14 includes a cylindrical section 36 which provides a substantially level surface and therefore a clarifying zone 38 for the pond 22 in use, and a frustoconical section 40 which provides an angled surface and therefore a drying or beach zone 42 for solids 44 by inhibiting the pond 22 from reaching the top of the frustoconical section 40 proximate the solids outlet 34 during use.

The decanter centrifuge 10 is able to separate solids from a solution by the scroll 12 gradually gathering and moving the solids 44 laterally toward the solids outlet 34 due to the speed differential between the scroll 12 and the bowl 14. The bowl g-force allows for a substantially liquid portion of a solution to be disposed proximate the internal surface 18 of the bowl 14 and the solids 44 to be disposed further from the internal surface 18 of the bowl 14 in order to be more easily collected by the flights 26 of the scroll 12.

The person skilled in the art will understand that the decanter centrifuge 10 is not limited to this particular embodiment of a decanter centrifuge and may include various different components in order to function to separate solids in a manner substantially similar as described.

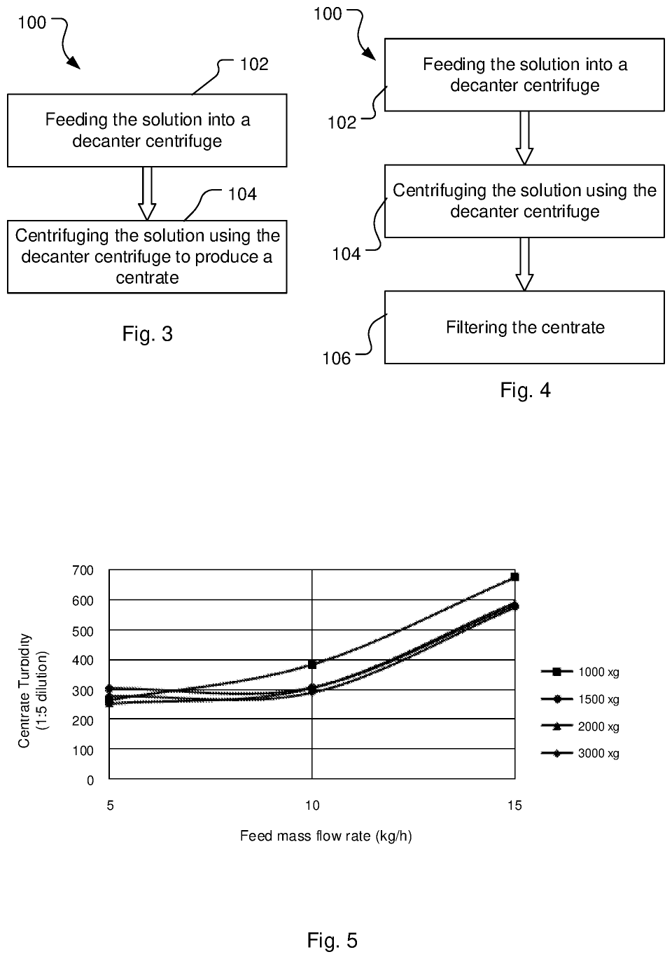

FIG. 3 shows an embodiment of a method 100 of separating solids from a solution 46 derived from blood plasma. The method 100 comprises feeding 102 a solution 46 into a decanter centrifuge, such as the decanter centrifuge 10, at a feed rate which may be measured in kilograms per hour; and centrifuging 104 the solution 46 using the decanter centrifuge 10 to produce a centrate 48. The centrate 48 has a reduced solids content with respect to the solution 46 fed into the decanter centrifuge 10. In some embodiments, one or more of the feed rate, the scroll speed, the bowl g-force, the speed differential, and the weir dimension 20 are selected such that a mass of the centrate 48 is at least 75% of a mass of the liquid phase of the solution 46 fed into the decanter centrifuge 10.

The feeding 102 may be performed using a pump, such as a peristaltic pump. The feed rate of the solution 46 to the decanter centrifuge will depend on the size and capacity of decanter centrifuge. The feed rate of the solution may be up to to about 10,000 kilograms per hour (kg/h). In some embodiments the feed rate of the may be about 0.5 kg/h, about 1 kg/h, about 25 kg/h, about 50 kg/h, about 75 kg/h, about 100 kg/h, about 150 kg/h, about 200 kg/h, about 250 kg/h, about 300 kg/h, about 400 kg/h, about 450 kg/h, about 500 kg/h, about 600 kg/h, about 700 kg/h, about 800 kg/h, about 900 kg/h, about 1,000 kg/h, about 1,250 kg/h, about 1,500 kg/h, about 1,750 kg/h, about 2,000 kg/h, about 2,250 kg/h, about 2,500 kg/h, about 2,750 kg/h, about 3,000 kg/h, about 3,250 kg/h, about 3,500 kg/h, about 3,750 kg/h, about 4,000 kg/h, about 4,500 kg/h, about 5,000 kg/h, about 5,500 kg/h, about 6,000 kg/h, about 6,500 kg/h, about 7,000 kg/h, about 7,500 kg/h, about 8,000 kg/h, about 9,000 kg/h, or about 10,000 kg/h. The feed rate of the solution may be in a range between any two of the above listed feed rates.

The bowl g-force may be about 500 g, about 600 g, about 700 g, about 800 g, about 900 g, about 1000 g, about 1250 g, about 1500 g, about 1750 g, about 2000 g, about 2250 g, about 2500 g, about 2750 g, about 3000 g, about 3250 g, about 3500 g, about 3750 g, about 4000 g, about 4500 g, about 5000 g, about 5500 g, about 6000 g, about 6500 g, about 7000 g, about 7500 g, about 8000 g, about 8500 g, about 9000 g, about 9500 g, or about 10000 g. In an embodiment, the bowl g-force may be in a range between any two of the above upper and/or lower concentrations, such as about 1000 g to about 10000 g, for example from about 1000 g to about 4000 g.

The relationship between the bowl g-force and the bowl speed is parabolic and is directly proportional to a radius of the bowl 14 according to the following formula:

$$g = 1.118 \times 1 {0}^{- 5} \times R \times {S}^{2}$$

Where: g is the bowl g-force
   R is the bowl radius (centimetres)
   S is the bowl speed (rpm)

In the decanter centrifuge 10, the frustoconical section 40 proximate the solids outlet 34 of the bowl 14 has a radius of 20 mm (2 cm) and the cylindrical section 36 of the bowl 14 has a radius of 30 mm (3 cm). Thus, the reported g-force values are the g-forces in the cylindrical section 36 as these are the maximum g-forces within the bowl 14.

The speed differential between the scroll 12 and the bowl 14 may be from about 1 rpm to about 200 rpm, for example from about 1 rpm to about 100 rpm. The speed differential may be about 0.5 rpm, about 1 rpm, about 1.5 rpm, about 2 rpm, about 2.5 rpm, about 5 rpm, about 7.5 rpm, about 10 rpm, about 15 rpm, about 20 rpm, about 25 rpm, about 30 rpm, about 35 rpm, about 40 rpm, about 45 rpm, about 50 rpm, about 55 rpm, about 60 rpm, about 65 rpm, about 70 rpm, about 75 rpm, about 80 rpm, about 85 rpm, about 90 rpm, about 95 rpm, about 100 rpm, about 110 rpm, about 120 rpm, about 130 rpm, about 140 rpm, about 150 rpm, about 160 rpm, about 170 rpm, about 180 rpm, about 190 rpmor about 200 rpm. The speed differential may be in a range between any two of the above values. However, it can be appreciated that in some cases the speed differential between the scroll and bowl can exceed these values in case of larger centrifuges.

Higher speed differentials generally decrease the residence time as the solids 44 are being moved towards the solids outlet 34 by the flights 26 of the scroll 12 at a faster speed, which is referred to as “axial transport velocity”. However, a faster axial transport velocity can lead to a lower recovery due to less dewatering of the solids 44, otherwise known as a cake (i.e. a wetter cake). Conversely, a slower axial transport velocity can lead to an increased residence time, a higher recovery due to more dewatering of the cake, and a build-up of the solids 44 within the decanter which may lead to plugging and decanter shut down. It will be appreciated that the selection of speed differential will be dependent on a number of factors, for example the type and solid content of the feed, desired properties of the centrate, as well as striking a balance between a suitable operating efficiency and a suitable recovery for the system. The axial transport velocity can be calculated according to the following formula:

$$v = \frac{GN}{2 π}$$

Where: v is axial transport velocity (metres/minute)
   G is scroll pitch (m)
   N is differential speed (rpm)

The weir 16 may have an aperture of any suitable shape to provide the desired pond depth of the pond 22, for example, the weir 16 may have a circular, elliptical, or polygonal aperture. In some embodiments, the weir 16 has a circular aperture and has an annular shape. In these embodiments and as shown in FIG. 2, the weir dimension 20a, 20b, 20c is the diameter of the circular aperture, such that a larger weir dimension (e.g. 20c) provides a shallower pond depth (e.g. 22c) and a smaller weir dimension (e.g. 20a) provides a deeper pond depth (e.g. 22a).

The weir dimension 20 is selected such that the pond depth is from about 1 mm to about 100 mm. FIG. 2 shows three lab-scale example weirs 16a, 16b, 16c, which provide pond depths of 8 mm, 6 mm and 4 mm, respectively and will be discussed in greater detail below in the examples. It will be understood that the weir dimension 20a, 20b, 20c may be selected such that the pond depth is about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 22.5 mm, about 25 mm, about 27.5 mm, about 30 mm, about 935 mm, about 40 mm, about 45 mm, about 50 mm, about 55 mm, about 60 mm, about 65 mm, about 70 mm, about 75 mm, about 80 mm, about 90 mm, or about 100 mm. The weir dimension may be in a range between any two of the above values.

The pond depth, and therefore the filling volume in the pond 22 referred to as the “dewatering volume”, is directly proportional to the time that the solution 46 is in the centrifuge 10 before the centrate 48 is produced. This time is referred to as “residence time” and will be discussed in the examples section below. Residence time is governed by an inverse relationship with feed flow rate of the solution 46 fed into the decanter centrifuge 10, according to the following formula:

$${R}_{t} = \frac{V}{{Q}_{f}}$$

Where: Rₜ is residence time (seconds)
   V is the dewatering volume (litres)
   Q_{f} is the feed flow rate (litres per second)

The method 100 may provide the centrate 48 with a recovery of at least 75%. In other words, the mass of the centrate 48 may be at least 75% of the mass of the solution 46 fed into the decanter centrifuge 10. In some embodiments, the method may provide a centrate 48 with a recovery of at least 90%, or even at least 95%. Increasing recovery will provide a drier cake, which is indicative of less liquid product lost in the cake. Operating parameters such as feed rate, bowl g-force, speed differential and/or pond depth, as well as parameters of the solution 46, may be selected to increase recovery. Recovery may also be increased by the method 100 further comprising washing the solids 44 during the centrifuging. It will be understood that the recovery of the centrate 48 may be at least about 20%, at least about 25%, at least about 30%, at least about 35%, at least about 40%, at least about 45%, at least about 50%, at least about 55%, 60%, at least about 62.5%, at least about 65%, at least about 67.5%, at least about 70%, at least about 72.5%, at least about 75%, at least about 77.5%, at least about 80%, at least about 82.5%, at least about 85%, at least about 87.5%, at least about 90%, at least about 92.5%, or at least about 95%.

The method 100 may be performed such that a turbidity of the centrate is less than 400 (NTU). In some embodiments, a lower turbidity of the centrate 48 can be a general indicator of a higher recovery of the centrate 48 and increased separation of solids, therefore it is desirable for the centrate 48 to have a low turbidity. Operating parameters such as feed rate, bowl g-force, speed differential and/or pond depth, as well as parameters of the solution 46, may be selected to lower turbidity. It will be understood that the turbidity of the centrate 48 may be less than about 600NTU, less than about 550NTU, less than about 500NTU, less than about 450NTU, less than about 400NTU, less than about 375NTU, less than about 350NTU, less than about 325NTU, less than about 300NTU, less than about 275NTU, less than about 250NTU, less than about 225NTU, less than about 200NTU, less than about 175NTU, less than about 150NTU, less than about 125NTU, less than about 100NTU, less than about 75NTU, less than about 50NTU, less than about 35NTU, less than about 30NTU, or less than about 25NTU. The turbidity of the centrate 48 may be in a range between any two of the above values.

In some embodiments, the solution 46 comprises a blood plasma fraction, such as Fraction (I+)II+III comprising IgG, Fraction IV-4 comprising hemopexin, and Fraction V comprising albumin. The person skilled in the art will understand that the solution 46 is not limited to the use of blood plasma fractions, and that the blood plasma fractions are not limited to the aforementioned fractions and may include any suitable plasma fraction, such as any of those stated in the background section. The person skilled in the art will also understand that the solution 46 may be derived from other sources of blood such as animal blood, for example, bovine blood.

The use of different blood plasma fractions may require selecting different operating parameters of the decanter centrifuge 10 in order to obtain a higher recovery and a lower turbidity of the centrate 48. Also, certain blood plasma fractions may provide a higher recovery and a lower turbidity than other blood plasma fractions under the same operating parameters. For example, the use of Fraction V comprising albumin may provide a higher recovery and a lower turbidity as seen in the examples section.

The temperature of the solution 44 may be any suitable temperature for the composition of the solution. In an embodiment, the solution is maintained at a temperature in the range of from about 4° C. to about 37° C. In some embodiments, the temperature of the solution is less than about 25° C.

Preferably, the conductivity of the solution is from 2 mS/cm to about 40 mS/cm. For example, the conductivity of the solution may be about 2 mS/cm, 3 mS/cm, 4 mS/cm, 5 mS/cm, 6 mS/cm, 7 mS/cm, 8 mS/cm, 9 mS/cm, 10 mS/cm, 11 mS/cm, 12 mS/cm, 13 mS/cm, 14 mS/cm, 15 mS/cm, 16 mS/cm, 17 mS/cm, 18 mS/cm, 19 mS/cm, 20 mS/cm, 21 mS/cm, 22 mS/cm, 23 mS/cm, 24 mS/cm, 25 mS/cm, 26 mS/cm, 27 mS/cm, 28 mS/cm, 29 mS/cm, 30 mS/cm, 31 mS/cm, 32 mS/cm, 33 mS/cm, 34 mS/cm, 35 mS/cm, 36 mS/cm, 37 mS/cm, 38 mS/cm, 39 mS/cm, or 40 mS/cm. While typically, the conductivity of the solution is measured at room temperature it will be appreciated that the conductivity may also be measured at the given temperature of the solution being fed to the decanter centrifuge. In an embodiment, the solution may have a conductivity of from about 8 mS/cm to about 15 mS/cm when measured at room temperature.

The solution 46 may further comprise a buffer. In these embodiments, buffer is added to a product derived from blood plasma to form the solution 46 prior to being fed to the decanter centrifuge. The purpose of the buffer is to resuspend the blood plasma fraction, which is a precipitate and may be in the form of a paste, so that the solution 46 has a suitable viscosity to be fed into the decanter centrifuge 10. Any suitable buffer may be used, for example the buffer may comprise sodium acetate, water (e.g. water for injection (WFI)), or a phosphate, such as disodium phosphate.

The conductivity of the solution may be adjusted as required to achieve the desired conductivity. Where the solution comprises a buffer solution, the concentration of the buffer may be adjusted to achieve the desired conductivity without substantial dilution of the sample.

The ratio of blood plasma fraction to buffer used in the solution 46 may affect the recovery and/or turbidity of the centrate. This ratio is termed an extraction ratio of the solution 46 and is defined by kilograms of the blood plasma fraction to litres of the buffer. It will be appreciated that the extraction ratio may be varied depending on the fraction of plasma being used as well as practical considerations such as the space required to store and use the volume of buffer required for higher extraction ratios. For example, extraction ratios of 1:1 or higher may be used, but for a higher recovery at least an extraction ratio of 1:2 would be suitable. A practical maximum extraction ratio would be 1:20, though it will be understood by the person skilled in the art that the extraction ratio may exceed 1:20 if required to achieve a particular recovery and/or to resuspend a particular blood plasma fraction.

Considering these factors, the solution 46 may have an extraction ratio of from about 1:2 to about 1:10. However, the extraction ratio may be any suitable extraction ratio, for example the extraction ratio may be about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, about 1:10, about 1:10.5, about 1:11, about 1:11.5, about 1:12, about 1:12.5, about 1:13, about 1:13.5, about 1:14, about 1:14.5, about 1:15, about 1:15.5, about 1:16, about 1:16.5, about 1:17, about 1:17.5, about 1:18, about 1:18.5, about 1:19, about 1:19.5, about 1:20, or about 1:20.5.

In some embodiments, the solution 46 comprises a filter aid. In these embodiments, the blood plasma fraction, which is a precipitate and may be in the form of a paste, comprises a filter aid. The filter aid may be any suitable filter aid, for example the filter aid may be cellulose based or silica based.

In some embodiments, for example where the solution 46 comprises IgG, the method 100 further comprises adding octanoic acid to the solution 46 for delipidating the solution 46. In other embodiments, octanoic acid is added to the centrate 48 for delipidating the centrate 48. Where octanoic acid is used, calcium phosphate may be added to the solution 46 or the centrate 48 to neutralise any excess octanoic acid.

As shown for example in FIG. 3, the method 100 may further comprise filtering 106 the centrate 48. In some embodiments, the filtering 106 comprises using a depth filter (not shown) to filter the centrate 48. The filtering 106 may further include adding a filter aid to the centrate 48 prior to using the depth filter to filter the centrate 48. The filter aid may also instead or additionally be used to pre-coat the depth filter. The depth filter may have a retention rating of 15 microns or finer, for example a retention rating of between 6 microns and 15 microns, between 3 microns and 6 microns, between 1 micron and 3 microns, or between 0.4 microns and 0.8 microns. It will be appreciated that the retention rating refers to the pore size of the depth filter, with the upper value of the retention rating range representing the larger pore size proximate an outlet of the depth filter and the lower value of the range representing the smaller pore size proximate an outlet of the depth filter.

In some embodiments, the filtering 106 comprises using a membrane filter (not shown) to filter the centrate 48. The membrane filter is a 1 micron filter or finer, for example, a 1 micron filter, a 0.5 micron filter, a 0.22 micron filter, or a 0.2 micron filter.

A combination of the depth filter and the membrane filter may also be used to filter the centrate 48, preferably using the depth filter to filter the centrate 48 then using the membrane filter to filter the filtered centrate. It will be understood by the person skilled in the art that other filtering mediums and techniques may be used alternatively or in addition to the depth filter and/or the membrane filter. It will also be understood that the centrate 48 or the filtered centrate using the depth filter and/or the membrane filter constitutes a blood plasma product produced using the method 100.

Where the filtering 106 is included in the method 100, properties and operation of the filters, such as filter type, filter pore size and/or retention rating, may be selected in addition to operating parameters of the decanter centrifuge 10 such as feed rate, bowl g-force, speed differential and/or pond depth, as well as parameters of the solution 46, to achieve a desired recovery of the filtered centrate.

The recovery of the centrate 48 may be determined after the filtering 106 with the depth filter. That is, the recovery is defined as the mass of the filtered centrate as a percentage of the mass of the solution 46 fed into the decanter centrifuge 10. In embodiments where the filtering 106 comprises one or more filtering processes, the recovery of the filtered centrate after any one of said filtering processes may be at least 60%, for example at least 75%, at least 90%, at least 95%. In some examples, the recovery of filtered centrate may be at least about 20%, at least about 25%, at least about 30%, at least about 35%, at least about 40%, at least about 45%, at least about 50%, at least about 55%, 60%, at least about 62.5%, at least about 65%, at least about 67.5%, at least about 70%, at least about 72.5%, at least about 75%, at least about 77.5%, at least about 80%, at least about 82.5%, at least about 85%, at least about 87.5%, at least about 90%, at least about 92.5%, or at least about 95%.

Where the filtering 106 is included in the method 100, properties and operation of the filters, such as filter type and/or retention rating, may be selected in addition to operating parameters of the decanter centrifuge 10 such as feed rate, bowl g-force, speed differential and/or pond depth, as well as parameters of the solution 46, to achieve a desired turbidity of the filtered centrate. The turbidity of the filtered centrate may be assessed after one or more filtering stages. Operating parameters such as feed rate, bowl g-force, speed differential and/or pond depth, parameters of the solution 46, as well as filter type and/or retention rating, may be selected to lower turbidity.

For example, the turbidity of the filtered centrate using one or more filters may be less than about 600NTU, less than about 550NTU, less than about 500NTU, less than about 450NTU, less than about 400NTU, less than about 375NTU, less than about 350NTU, less than about 325NTU, less than about 300NTU, less than about 275NTU, less than about 250NTU, less than about 225NTU, less than about 200NTU, less than about 175NTU, less than about 150NTU, less than about 125NTU, less than about 100NTU, less than about 75NTU, less than about 50NTU, less than 35NTU, less than 30NTU, or less than about 25NTU.

In some embodiments, the centrate 48 is produced such that the centrate 48 is filterable with a throughput of at least 100 litres of the centrate per square metre (L/m²) of the one or more filters. Operating parameters such as feed rate, bowl g-force, speed differential and/or pond depth, parameters of the solution 46, as well as filter type, filter pore size and/or retention rating, may be selected to achieve a desired throughput. For example the centrate 48 may be filterable in one or more filter stages with a throughput of at least about 25 L/m², at least about 50 L/m², at least about 75 L/m², at least about 100 L/m², at least about 125 L/m², at least about 150 L/m², at least about 175 L/m², at least about 200 L/m², at least about 225 L/m², at least about 250 L/m², at least about 275 L/m², at least about 300 L/m², at least about 325 L/m², at least about 350 L/m², at least about 375 L/m², or at least about 400 L/m², at least about 450 L/m², at least about 500 L/m², or at least about 600 L/m², or at least about 700 L/m², or at least about 800 L/m², or at least about 900 L/m², or at least about 1000 L/m², or at least about 1100 L/², or at least about 1200 L/m², or at least about 1300 L/m². The centrate 48 may be filterable in one or more filter stages with a throughput in a range between any two of the above values. It will be appreciated that the throughput can be scaled by the addition of additional filter units.

Advantageously, the use of the decanter centrifuge in the method 100 separates solids from the solution 46 to an extent comparable to existing techniques such as a filter press, while providing a reduced footprint and a faster overall process. It will be appreciated that operation of the decanter centrifuge is also less labour intensive than the filter press and does not require the emptying of individual filter plates and the replacing of filter sheets which adds to the total processing time.

In some embodiments, the use of the decanter centrifuge 10 in the method 100 provides a recovery of at least 75%, thereby providing a comparable recovery to the use of a filter press. In the embodiments of the method 100 comprising filtering the centrate 48, the filter used provides a throughput of at least 100 L/m², which is a comparable throughput to the filter press, thus indicating that the embodiment of the method 100 including the filtering 106 may be as effective and fast as the filtration using the filter press, while providing the advantages of a reduced footprint and labour as stated above.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

The following examples are to be understood as illustrative only. The following examples should therefore not be construed as limiting the embodiments of the disclosure in any way.

FIGS. 5-16 show results of experiments conducted with respect to various embodiments of the method 100 to separate solids from the solution 46.

### Example 1—Effects of Varying the Operating Parameters Such as Feed Flow Rate, Bowl g-Force, Speed Differential, and Weir Dimension

FIG. 2 shows a schematic comparing three annular weirs 16a, 16b, 16c with different weir dimensions 20a, 20b, 20c, which in this embodiment are inner diameters of the weir. Indications of the corresponding pond depths 22a and 22c for dimensions 20a and 20c, respectively, and how the different pond depths affect the clarifying zone 38a, 38c and drying zone 42a, 42c lengths are also shown in FIG. 2. In the present examples, the three annular weirs 16a, 16b, 16c have inner diameters of 44 mm, 48 mm and 52 mm that defined pond depths 22 of 8 mm, 6 mm and 4 mm, respectively.

Experiments were conducted which indicated that a lower pond depth due to an increased inner weir diameter provided a shorter clarifying zone 38 and a larger drying zone 42, thereby leading to a smaller filling volume and a shorter residence time. This led to a lower centrate clarity. Conversely, a higher pond depth due to an decreased inner weir diameter provided a longer clarifying zone 38 and a shorter drying zone 42, thereby leading to a larger filling volume and a longer residence time. This led to a higher centrate clarity, and a similar recovery compared with that from the lower pond depth, which is why the higher pond depth of 8 mm was preferred for the following examples.

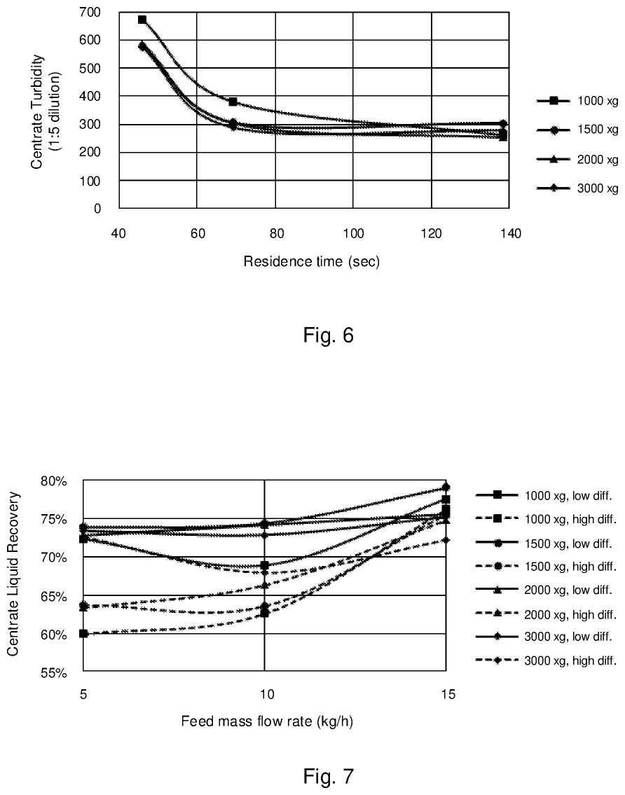

FIG. 5 shows a graph of centrate turbidity versus feed flow rate under different bowl g-forces with a hemopexin extract (Fraction IV-4) at an extraction ratio of 1:2.5 (3 kg of Fraction IV-4:7.5 L of 40 mM disodium phosphate buffer), a speed differential of the lowest possible (10, 15, 20 or 30 rpm depending on the other operating parameters) and a pond depth of 8 mm. Experimental results from FIG. 5 are represented in FIG. 6 by converting the feed flow rate to residence time. When the feed flow rate increased from 5 to 15 kg/h, the residence time decreased from 138 sec to 46 sec. The graphs shown in FIGS. 5 and 6 indicate that regardless of the bowl g-force, as the feed flow rate increased so did the centrate turbidity in most cases.

FIG. 7 shows a graph of recovery versus feed flow rate under different bowl g-force and speed differential combinations with a hemopexin extract at an extraction ratio of 1:2.5 and a pond depth of 8 mm. The graph shown in FIG. 7 indicates that the centrate liquid recovery is inversely correlated to differential speed and directly correlated to the bowl g-force.

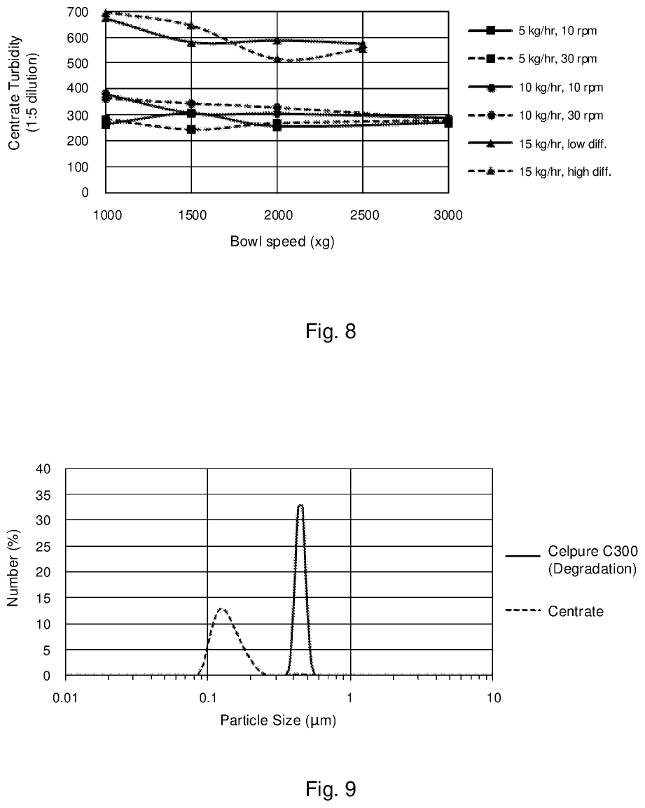

FIG. 8 shows a graph of centrate turbidity versus bowl g-force under different feed flow rate and speed differential combinations with a hemopexin extract (Fraction IV-4) at an extraction ratio of 1:2.5 (3 kg of Fraction IV-4:7.5 L of 40 mM disodium phosphate buffer) and a pond depth of 8 mm. The graph shown in FIG. 8 indicates a decline trend of turbidity when the bowl speed increased, with higher differential speed normally leading to higher centrate turbidity.

In this experiment, it was found that increasing the bowl g-force to 1500 g or above caused a high volume of foam in the centrate 48, which is generally undesirable. It will be understood that the foam was produced under the particular conditions of this experiment including the composition of the solution 46 and the decanter design, and that such an undesirable result does not preclude the use of higher bowl g-forces in the method 100.

An experiment of the method 100 was conducted with the hemopexin extract (Fraction IV-4) at an extraction ratio of 1:2.5 (3 kg of Fraction IV-4:7.5 L of 40 mM disodium phosphate buffer) using the optimized operation parameter combination, which yielded a recovery of 76% in the centrate 48 as measured by high-performance liquid chromatography (HPLC).

### Example 2—Particle Size Distribution in the Centrate

FIG. 9 shows a particle size distribution analysis using dynamic light scattering of the centrate 48 produced from an experiment with a hemopexin extract (Fraction IV-4) at an extraction ratio of 1:2.5 (3 kg of Fraction IV-4:7.5 L of 40 mM disodium phosphate buffer), a bowl g-force of 1000 g, a pond depth of 8 mm, a speed differential of 10 rpm, and a feed flow rate of 5 kg/h. The distribution shown in FIG. 9 demonstrates that in this experiment, the decanter centrifuge 10 separated the majority of the particles greater than 0.23 microns from the solution 46 and that the remaining particles larger than 0.23 microns were degraded filter aid, in this case Celpure® C300B.

### Example 3—Comparing the Effects of Depth Filters with and without Filter Aids

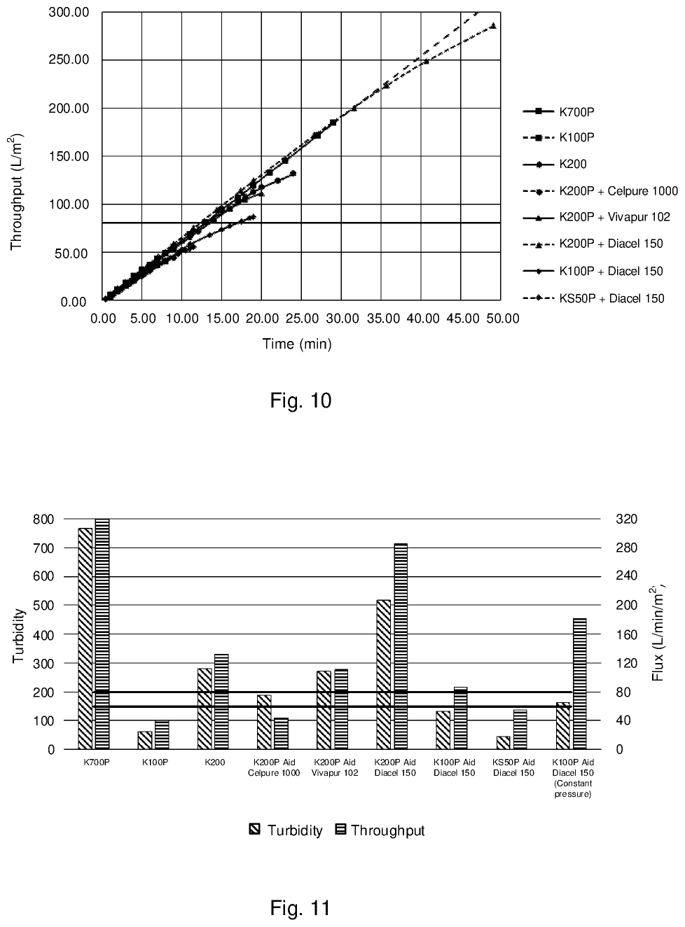

FIG. 10 shows a graph of throughput versus time to compare the use of different depth filter media with and without filter aids to filter the centrate 48. In this experiment, the centrate 48 was produced from a hemopexin extract (Fraction IV-4) at an extraction ratio of 1:2.5 (3 kg of Fraction IV-4:7.5 L of 40 mM disodium phosphate buffer) using the decanter centrifuge 10 with a bowl g-force of 1000 g, a pond depth of 8 mm, a speed differential of 10 rpm, and a feed rate of 5 kg/h. The centrate 48 was filtered using each depth filter/depth filter and filter aid combination for a period of time until the pressure limit has been reached. The graph shown in FIG. 10 also includes a minimum viable throughput threshold of 80 L/m² selected by the Applicant, and as shown, the Seitz® K700 P depth filter media, Seitz® K200 depth filter media, Seitz® K200 P depth filter media pre-coated with Vivapur® 102 filter aid, Seitz®K200 P depth filter media pre-coated with Diacel® 150 filter aid, and Seitz®K100 P depth filter media pre-coated with Diacel® 150 filter aid surpassed the minimum viable throughput threshold.

FIG. 11 shows a column graph additionally comparing the turbidity of filtrates produced from the experimental results shown in FIG. 10. The graph of FIG. 11 shows the minimum viable throughput threshold from FIG. 10 and also includes a maximum viable turbidity threshold of 150NTU selected by the Applicant. As shown, the combination of the Seitz® K100 P depth filter media and the Diacel® 150 (K100 P+D150) filter aid was the only combination that met both the minimum viable throughput threshold and the maximum viable turbidity threshold and was therefore selected for use in further experiments.

### Example 4—Comparing the Throughput of Depth Filtration Post Decanter Centrifuge to the Filter Press

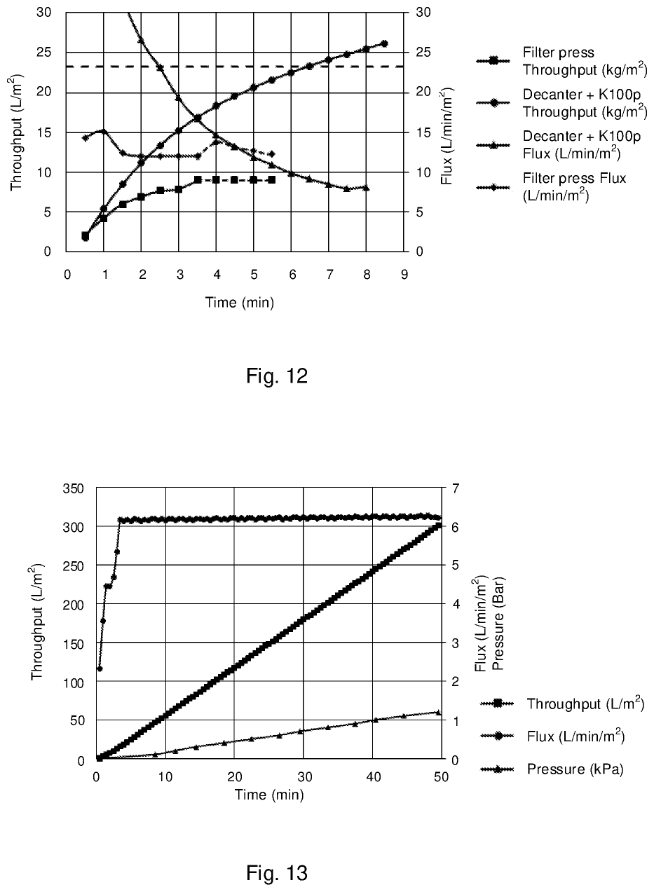

FIG. 12 shows a graph of throughput and flux versus time to compare the results of the K100 P+D150 versus a 3M® 90SP filter pre-coated with Celpure® 1000 which aimed to mimic the use of a filter press for filtering solids from the solution 46 derived from blood plasma. For the K100 P+D150, the solution 46 was a hemopexin extract (Fraction IV-4) at an extraction ratio of 1:5 (1.5 kg of Fraction IV-4:7.5 L of phosphate buffer) and the decanter centrifuge 10 had a bowl g-force of 1000 g, a pond depth of 8 mm, a speed differential of 5 rpm, and a feed rate of 5 kg/h. The extraction ratio was increased to 1:5 in this experiment, which yielded a recovery of 85% determined by HPLC, compared to the 76% yield observed when using the 1:2.5 extraction ratio in the previous examples. A hemopexin extract (Fraction IV-4) at an extraction ratio of 1:2.5 (3 kg of Fraction IV-4:7.5 L of 40 mM disodium phosphate buffer) was filtered using the 3M® 90SP filter pre-coated with Celpure® 1000.

The throughput of the K100 P+D150 was higher than that of the mimicked filter press and surpassed the goal throughput value selected by the Applicant of 23.2 kilograms of blood plasma fraction precipitate per square metre of filter (kg/m²). This goal was calculated based on the actual filter press producing an expected throughput of 9.08 kg/m² with a total filter area of 51 m², whereas the K100 P+D150 only had a total filter area of 20 m² and thus, there is a 2.5-fold increase in the throughput of the actual filter press. The flux of the K100 P+D150 did fall, after approximately 7.5 minutes, to a flux of 8 L/min/m². However, this flux was still higher than the expected 6.25 L/min/m² flux of the actual filter press.

### Example 5—Comparing the Throughput of the Membrane Filter Post Depth Filtration Between Decanter Centrifuge and Filter Press Routes

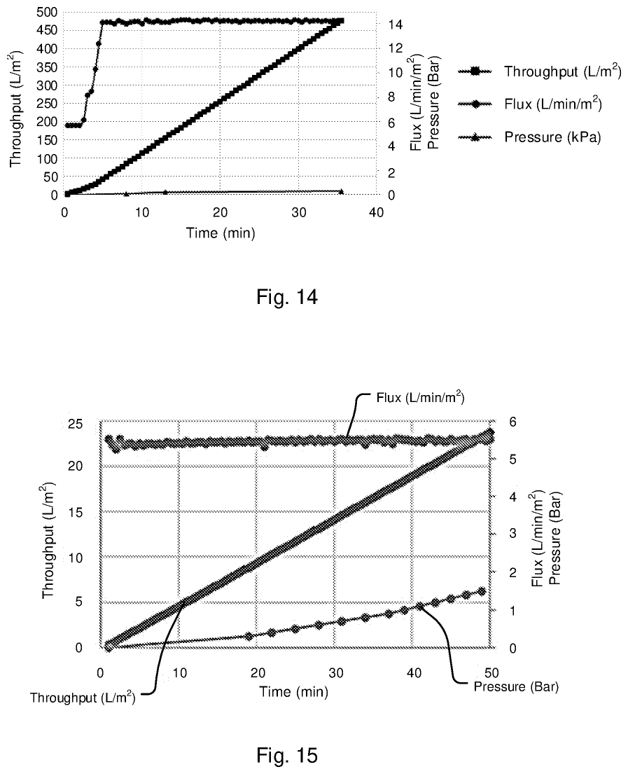

FIGS. 13 and 14 show the results of the filtrate from the K100 P+D150 being subsequently filtered using a membrane filter. The centrate 48 filtered using the K100 P+D150 had a hemopexin extract (Fraction IV-4) at an extraction ratio of 1:5 (1.5 kg of Fraction IV-4:7.5 L of phosphate buffer) and the decanter centrifuge 10 had a bowl g-force of 1000 g, a pond depth of 8 mm, a speed differential of 5 rpm, and a feed rate of 5 kg/h. The extraction ratio was increased to 1:5 in this experiment, which yielded a recovery of 85% determined by HPLC, compared to the 76% yield observed when using the 1:2.5 extraction ratio in the previous examples.

FIG. 13 shows a graph of throughput and flux versus time using the Millipak® 20 0.22 micron filter, which matched the expected 303 L/m² throughput using the same membrane filter to subsequently filter the filter press filtrate. The flux also remained approximately constant throughout the filtration process.

FIG. 14 shows a graph of throughput and flux versus time using the Milligard® 0.5/0.2 micron filter, which reached a throughput of 480 L/m², exceeding the expected 303 L/m² throughput using the Millipak® 20 0.22 micron filter to subsequently filter the filter press filtrate. The flux also remained approximately constant throughout the filtration process.

### Example 6—Effects of Increasing the Extraction Ratio

FIG. 15 shows a graph of throughput and flux versus time using the K100 P+D150 to filter a centrate 48 produced from a hemopexin extract (Fraction IV-4) at an extraction ratio of 1:10 (0.5 kg of Fraction IV-4:5 L of phosphate buffer) and the decanter centrifuge 10 had a bowl g-force of 1000 g, a pond depth of 8 mm, a speed differential of 5 rpm, and a feed rate of 5 kg/h. Similarly to the results shown in FIG. 12, the throughput surpassed the goal throughput value of 23.2 kg/m², and additionally, this increased extraction ratio allowed for a more stable flux than that in the experimental results of FIG. 14, with no observable flux decay over time.

The recovery of the centrate 48 was 90% and the recovery of the K100 P+D150 filtration was 95% as measured by HPLC, which results in an overall recovery of 85% from the centrifuging and filtering. The turbidity of the centrate 48 was 390NTU and the turbidity of the filtrate following the K100 P+D150 was 56.9NTU.

### Example 7—Applying the Method to a Solution Comprising IgG

An experiment of the method 100 was conducted with a IgG extract (Fraction I+II+III) at an extraction ratio of 1:4.5 (0.9 kg of Fraction I+II+III: 4.05 L of 0.22M sodium acetate buffer) and was incubated for 7 hours at room temperature once the solution 46 reached 20° C. The decanter centrifuge 10 had a bowl g-force of 1000 g, a pond depth of 8 mm, a speed differential of 2 rpm, and a feed rate of 5 kg/h.

Prior to the centrifuging 104, a delipidating reagent was added to the solution 46, in this case 83 g of octanoic acid was added over 40 minutes and the solution 46 was incubated at room temperature for 6 hours. Following this, 24.7 g of calcium phosphate was added to the solution 46 to neutralise any excess octanoic acid prior to decanting, and the solution 46 was incubated for a further 75 minutes at room temperature.

The recovery of the centrate 48 following the centrifuging 104 was 77% as measured using spectrophotometric quantitation at 280 nm and the centrate turbidity was 1611NTU. This lower recovery is due to the high moisture in the cake. The cake was observed to be very sticky, most possibly due to the effect of octanoic acid. In-situ washing during the centrifuging 104 or increasing the extraction ratio may improve the recovery under the conditions of this experiment.

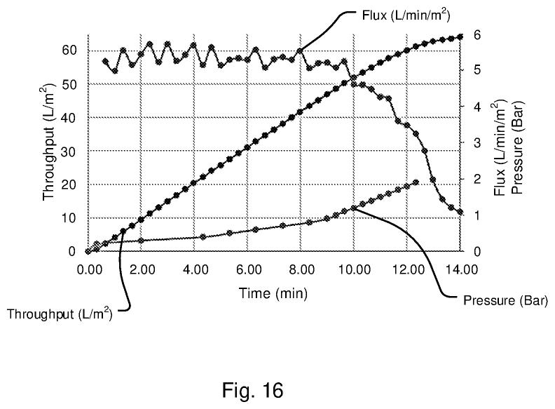

FIG. 16 shows a graph of throughput and flux versus time using the K100 P+D150 to filtrate a centrate 48 produced from a IgG extract (Fraction I+II+III) at an extraction ratio of 1:4.5 (0.9 kg of Fraction I+II+III: 4.05 L of 0.22M sodium acetate buffer) and was incubated for 7 hours at room temperature once the solution 46 reached 20° C. The decanter centrifuge 10 had a bowl g-force of 1000 g, a pond depth of 8 mm, a speed differential of 2 rpm, and a feed rate of 5 kg/h. The throughput reached 64 L/m² which is equivalent to 0.069 m²/kg blood plasma fraction (Fraction I+II+III), exceeding the expected result of 0.1 m²/kg using the filter press with the same blood plasma fraction.

In a further experiment, the centrate 48 was delipidated after the centrifuging 104 instead of delipidating the solution 46 prior to the centrifuging 104. A delipidating reagent was added to the centrate 48, in this case 68.9 g of octanoic acid was added over 40 minutes and the centrate 48 was incubated at room temperature for 400 minutes. Following this, 20.7 g of calcium phosphate was added to the centrate 48 to neutralise any excess octanoic acid prior to filtering, and the centrate 48 was incubated for a further 75 minutes at room temperature.

This experiment resulted in an increased recovery of 85% for decanter centrifuging compared to 77% achieved in the previous experiment due to less amount of precipitate in the solution 46. However, a lot of lipids were precipitated after octanoic acid addition. Thus, there should be another centrifugation or filtration step to remove all the lipids, which will cause a decrease in the overall yield.

In these experiments, it was found that increasing the bowl g-force to 1500 g or above caused a high volume of foam in the centrate 48, which is generally undesirable. It will be understood that the foam was produced under the particular conditions of this experiment including the composition of the solution 46 and the decanter design, and that such an undesirable result does not preclude the use of higher bowl g-forces in the method 100.

### Example 8—Applying the Method to a Solution Comprising Albumin

An experiment of the method 100 was conducted with an albumin extract (Fraction V) at an extraction ratio of 1:2 (1.6667 kg of Fraction V: 0.01M 3.3333 L of sodium acetate buffer) and incubated at room temperature for 2 hours. The decanter centrifuge 10 had a bowl g-force of 1000 g, a pond depth of 8 mm, a speed differential of 1 rpm, and a feed rate of 5 kg/h.

The recovery was found to be 97% as measured by a nephelometer and the turbidity was found to be 33.6NTU, which provided a very dry cake, indicating that almost no liquid product was lost in the cake. These results indicate that the decanter centrifuge 10 alone would provide an equal or even higher recovery than using the filter press for these parameters when separating solids from a solution 46 comprising albumin.

1. A method of separating solids from a solution derived from blood plasma, the method comprising:
feeding the solution into a decanter centrifuge at a feed rate; and
centrifuging the solution using the decanter centrifuge to produce a centrate having a reduced solids content with respect to the solution fed into the decanter centrifuge, wherein the decanter centrifuge comprises:
a scroll rotating at a scroll speed,
a bowl rotating at a bowl speed to produce a bowl g-force, wherein the bowl speed is different to the scroll speed, thereby defining a speed differential, and
a weir on an internal surface of the bowl, the weir having a dimension that defines a pond depth, and
wherein one or more of the feed rate, the scroll speed, the bowl g-force, the speed differential, and the weir dimension are selected such that a mass of the centrate is at least 75% of a mass of the liquid phase of the solution fed into the decanter centrifuge.

2. The method of claim 1, wherein the solution comprises a blood plasma fraction.

3. The method of claim 2, wherein the solution comprises a buffer.

4. The method of claim 3, wherein the buffer comprises sodium acetate, water for injection, or a phosphate.

5. The method of claim 3 or claim 4, wherein the solution has an extraction ratio of kilograms of blood plasma fraction to litres of buffer of from about 1:2 to about 1:10.

6. The method of any one of the preceding claims, wherein the solution comprises hemopexin.

7. The method of any one of claims 1-5, wherein the solution comprises albumin.

8. The method of any one of claims 1-5, wherein the solution comprises immunoglobulin G.

9. The method of claim 8, further comprising adding octanoic acid to the solution or the centrate for delipidation.

10. The method of any one of the preceding claims, wherein the solution comprises a filter aid.

11. The method of any one of the preceding claims, wherein the feed rate of the solution is up to 10,000 kilograms per hour.

12. The method of any one of the preceding claims, wherein the bowl g-force is from about 1000 g to about 4000 g.

13. The method of any one of the preceding claims, wherein the speed differential is from about 1 revolution per minute to about 200 revolutions per minute.

14. The method of any one of the preceding claims, wherein the weir dimension is selected such that the pond depth is from about 1 millimetres to about 100 millimetres.

15. The method of any one of the preceding claims, wherein the mass of the centrate is at least 75% of the mass of the solution fed into the decanter centrifuge.

16. The method of any one of the preceding claims, wherein the mass of the centrate is at least 95% of the mass of the solution fed into the decanter centrifuge.

17. The method of any one of the preceding claims, wherein a turbidity of the centrate is less than 400 nephelometric turbidity units (NTU).

18. The method of any one of the preceding claims, further comprising washing the solids during the centrifuging.

19. The method of any one of the preceding claims, further comprising filtering the centrate.

20. The method of claim 19, wherein the filtering the centrate comprises using a depth filter.

21. The method of claim 20, wherein the depth filter has a retention rating of 15 microns or finer.

22. The method of claim 20 or claim 21, wherein the centrate is produced such that the centrate is filterable with a throughput of at least 100 litres of the centrate per square metre of the depth filter.

23. The method of any one of claims 20-22, wherein a turbidity of the filtered centrate using the depth filter is less than 150NTU.

24. The method of any one of claims 20-23, wherein the mass of the filtered centrate using the depth filter is at least 75% of the mass of the centrate.

25. The method of any one of claims 20-24, wherein the mass of the filtered centrate using the depth filter is at least 95% of the mass of the centrate.

26. The method of any one of claims 20-25, further comprising adding a filter aid to the centrate or pre-coating the depth filter prior to the using the depth filter to filter the centrate.

27. The method of any one of claims 19-26, wherein the filtering the centrate comprises using a membrane filter or a combination of a depth filter and a membrane filter.

28. The method of claim 27, wherein the membrane filter is a 1 micron filter or finer.

29. The method of claim 27 or claim 28, wherein the filtrate is produced with a throughput of at least 300 litres per square metre of the membrane filter.

30. The method of any one of claims 27-29, wherein a turbidity of the filtered centrate using the membrane filter is less than 50NTU.

31. The method of any one of claims 27-30, wherein the mass of the filtered centrate using the membrane filter is at least 75% of the mass of the solution fed into the decanter centrifuge.

32. The method of any one of claims 27-31, wherein the mass of the filtrate using the membrane filter is at least 99% of the mass of the solution applied to the membrane filter.

33. The method of any one of the preceding claims, wherein the solution has a conductivity of about 2 mS/cm to about 40 mS/cm.

34. The method of any one of the preceding claims, wherein the solution has a conductivity of about 8 mS/cm to about 15 mS/cm.

35. The method of any one of the preceding claims, wherein the solution is at a temperature of from about 4° C. to about 37° C.

36. A method of separating solids from a solution derived from blood plasma, the method comprising:
feeding the solution into a decanter centrifuge at a feed rate; and
centrifuging the solution using the decanter centrifuge to produce a centrate having a reduced solids content with respect to the solution fed into the decanter centrifuge, wherein the decanter centrifuge comprises:
a scroll rotating at a scroll speed,
a bowl rotating at a bowl speed to produce a bowl g-force, wherein the bowl speed is different to the scroll speed, thereby defining a speed differential, and
a weir on an internal surface of the bowl, the weir having a dimension that defines a pond depth.

37. A blood plasma product produced using the method of any one of the preceding claims.
